(19) United States
(12) Patent Application Publication
(10) Pub. No.: US 2025/0049500 A1
(43) Pub. Date: Feb. 13, 2025

(54) ACCURATE POSITIONING AND SHAPE VISUALIZATION OF BALLOON CATHETER ABLATION TAGS

(71) Applicant: Biosense Webster (Israel) Ltd., Yokneam (IL)

(72) Inventors: Eid ADAWI, Tur'an (IL); Zvi DEKEL, Zichron Yaakov (IL)

(73) Assignee: Biosense Webster (Israel) Ltd., Yokneam (IL)

(21) Appl. No.: 18/931,771

(22) Filed: Oct. 30, 2024

Related U.S. Application Data
(63) Continuation of application No. 17/081,926, filed on Oct. 27, 2020, now Pat. No. 12,137,967.
(60) Provisional application No. 62/934,331, filed on Nov. 12, 2019.

(51) Int. Cl. A61B 18/14; A61B 18/00; A61B 90/00

(52) U.S. Cl. CPC A61B 18/1492; A61B 90/37; A61B 90/39; A61B 2018/0022; A61B 2018/00351; A61B 2018/00577; A61B 2018/00982; A61B 2090/374; A61B 2090/3762; A61B 2090/378; A61B 2090/3937

(57) ABSTRACT

Embodiments of the present invention include generating a 3D model of a body cavity, and receiving, from a position transducer associated with a medical probe configured to be inserted into the cavity and having at least one elongated electrode disposed along a distal end of the probe, signals indicating orientation and location coordinates of the distal end within the body cavity. Based on the model and the signals, while parts of the given electrode other than the identified segment are not in contact with the inner surface of the cavity, a segment along a length of a given electrode that is in contact with tissue at a site on an inner surface of the cavity is identified. A graphical representation of the model with a visual marker at a location on the model corresponding to the site contacted by the segment of the given electrode is rendered to a display.

### CROSS-REFERENCE TO RELATED APPLICATIONS

This application is a Continuation of prior U.S. patent application Ser. No. 17/081,926 filed Oct. 27, 2020 (Attorney Docket No.: BIO6119USNP1-253757.000177), which claims the benefit of U.S. Provisional Patent Application 62/934,331 filed on Nov. 12, 2019 (Attorney Docket No. BIO6119USPSP1), whose disclosure is incorporated herein by reference as if set forth in full herein.

### FIELD

The present invention relates generally to medical imaging, and specifically to visualizing an area of tissue in contact with an ablation electrode.

### BACKGROUND

Arrhythmias are abnormal heart rhythms that are typically caused by a small area of cardiac tissue that produces irregular heartbeats. Cardiac ablation is a medical procedure that can be performed to treat an arrhythmia by destroying the area of the cardiac tissue causing the irregular heartbeats.

In some instances, cardiac ablation can be performed using a balloon catheter. A balloon catheter comprises an inflatable balloon at its distal end that can be inflated and deflated as necessary. The balloon typically comprises multiple electrodes configured to deliver ablation energy to tissue in contact with the electrodes. The balloon is typically deflated while the catheter is inserted into a body cavity (e.g., a heart) of a patient, inflated in order to perform the necessary procedure, and deflated again upon completing the procedure.

U.S. Pat. No. 6,514,249 to Maguire et al., describes a positioning system for orienting an ablation element within a pulmonary vein ostium. The system includes a position monitoring assembly that can be used to position a circumferential ablation member along a circumferential region of tissue at a location where a pulmonary vein extends from a left atrium.

U.S. Patent Application 2008/0275300 to Rothe et al., describes a complex shape steerable tissue visualization and manipulation catheter. The catheter includes a steering mechanism that can adjust a position of a visualization hood or membrane through which underlying tissue may be visualized.

The description above is presented as a general overview of related art in this field and should not be construed as an admission that any of the information it contains constitutes prior art against the present patent application.

### SUMMARY

There is provided, in accordance with an embodiment of the present invention, an apparatus including an invasive medical probe configured to be inserted into a body cavity and including a distal end having at least one elongated electrode disposed along the distal end, a position transducer associated with the medical probe, a memory configured to store a three-dimensional (3D) model of the body cavity, a display, and a processor configured to receive, from the position transducer, signals indicative of orientation and location coordinates of the distal end within the body cavity, to identify, based on the 3D model and the signals, a segment along a length of a given elongated electrode that is in contact with tissue at a site on an inner surface of the body cavity, while parts of the given elongated electrode other than the identified segment are not in contact with the inner surface of the body cavity, and to render to the display a graphical representation of the 3D model with a visual marker at a location on the 3D model corresponding to the site contacted by the segment of the given elongated electrode.

In some embodiments, the at least one elongated electrode is disposed longitudinally along the distal end of the medical probe.

In an additional embodiment, the invasive medical probe may also include an inflatable balloon that extends from a lumen in the distal end of the medical probe. In one embodiment, the at least one elongated electrode is disposed longitudinally on a surface of the balloon.

In another embodiment, the apparatus may also include an ablation module configured to deliver ablation energy to the at least one elongated electrode, thereby ablating the tissue that is in contact with the at least one electrode. In one embodiment, the visual marker corresponds to the site ablated by the segment of the given elongated electrode.

In a supplemental embodiment, processor can be configured, to receive, prior to receiving the signals, 3D model data for the body cavity, and to generate, using the 3D model data, the 3D model. In some embodiments, wherein the 3D model data can be selected from a list consisting of anatomical mapping data, computed tomography data, magnetic resonance imaging data and ultrasound data.

In a further embodiment, the processor can be configured to determine, based on the 3D model and the signals, an engagement contour of the segment along the length of the given elongated electrode that is in contact with the tissue, and wherein the processor is configured to render the visual marker at a location on the 3D model by presenting, at the location on the 3D model, a visual marker contour corresponding to the engagement contour.

There is also provided, in accordance with an embodiment of the present invention, a method including generating a three-dimensional (3D) model of a body cavity, receiving, from a position transducer associated with a medical probe configured to be inserted into the body cavity and including a distal end having at least one elongated electrode disposed along the distal end, signals indicative of orientation and location coordinates of the distal end within the body cavity, identifying, based on the 3D model and the signals, a segment along a length of a given elongated electrode that is in contact with tissue at a site on an inner surface of the body cavity, while parts of the given elongated electrode other than the identified segment are not in contact with the inner surface of the body cavity, and rendering to the display a graphical representation of the 3D model with a visual marker at a location on the 3D model corresponding to the site contacted by the segment of the given elongated electrode.

There is further provided, in accordance with an embodiment of the present invention, a computer software product operated in conjunction with a medical probe configured to be inserted into a body cavity and including a distal end having at least one elongated electrode disposed along the distal end, the product including a non-transitory computer-readable medium, in which program instructions are stored, which instructions, when read by a computer, cause the computer to generate a three-dimensional (3D) model of a body cavity, to receive, from a position transducer associated with the medical probe, signals indicative of orientation and location coordinates of the distal end within the body cavity, to identify, based on the 3D model and the signals, a segment along a length of a given elongated electrode that is in contact with tissue at a site on an inner surface of the body cavity, while parts of the given elongated electrode other than the identified segment are not in contact with the inner surface of the body cavity, and to render to the display a graphical representation of the 3D model with a visual marker at a location on the 3D model corresponding to the site contacted by the segment of the given elongated electrode.

### BRIEF DESCRIPTION OF THE DRAWINGS

The disclosure is herein described, by way of example only, with reference to the accompanying drawings, wherein:

FIG. 1 is a schematic, pictorial illustration of a medical system comprising a medical console configured to generate a three-dimensional (3D) model of a body cavity, and a medical probe whose distal end comprises a balloon, in accordance with an embodiment of the present invention;

FIG. 2 is a schematic pictorial illustration of the distal end comprising multiple elongated electrodes mounted on the balloon, in accordance with an embodiment of the present invention;

FIG. 3 is a schematic cutaway view of the distal end of the medical probe, in accordance with an embodiment of the present invention;

FIG. 4 is a schematic pictorial illustration of voxels that can be used to generate a 3D model of the body cavity, in accordance with an embodiment of the present invention;

FIG. 5 is a block diagram showing an example of the 3D model of the body cavity, in accordance with an embodiment of the present invention;

FIG. 6 is a schematic pictorial illustration of voxels that can be used to generate a 3D model of the balloon, in accordance with an embodiment of the present invention;

FIG. 7 is a block diagram showing an example of the 3D model of the balloon, in accordance with an embodiment of the present invention;

FIG. 8 is a flow diagram that schematically illustrates a method of presenting, on the 3D model of the body cavity, visual markers that correspond to engagement areas where the elongated electrodes engage tissue in the body cavity, in accordance with an embodiment of the present invention;

FIGS. 9-11 are schematic pictorial illustrations showing examples of the elongated electrodes engaging tissue in the body cavity, in accordance with an embodiment of the present invention;

FIG. 12 is a schematic pictorial illustration of a graphical representation of the 3D model and the visual markers, in accordance with a first embodiment of the present invention;

FIG. 13 is a schematic pictorial illustration of the graphical representation of the 3D model and the visual markers, in accordance with a second embodiment of the present invention; and

FIG. 14 is a schematic pictorial illustration of the graphical representation of the 3D model and the visual markers, in accordance with a third embodiment of the present invention.

### DETAILED DESCRIPTION

### Overview

Embodiments of the present invention describe a system and a method for presenting visual markers indicating areas of tissue being treated during a medical procedure such as cardiac ablation. As described hereinbelow, the system comprises an invasive medical probe configured to be inserted into a body cavity and comprising a distal end having at least one elongated electrode disposed along the distal end. For example, the medical probe may comprise an intracardiac catheter having a balloon affixed to the distal end, wherein the one or more elongated electrodes are mounted on an outer surface of the balloon.

The system also comprises a position transducer associated with the medical probe. In some embodiments, as described hereinbelow, the position transducer may comprise a magnetic field sensor affixed to the distal end of the medical probe.

The system additionally comprises a display and a memory configured to store a three-dimensional (3D) model of the body cavity. In some embodiments, as described hereinbelow, the 3D model may be derived from an anatomical map, computerized tomography (CT) image data, magnetic resonance imaging (MRI) image data, or ultrasound image data.

The system further comprises a processor configured to receive, from the position transducer, signals indicative of orientation and location coordinates of the distal end within the body cavity, and to identify, based on the 3D model and the signals, a segment along a length of the elongated electrode that is in contact with tissue at a site on an inner surface of the body cavity, while parts of the elongated electrode other than the identified segment are not in contact with the inner surface of the body cavity. The processor is also configured to render to the display a graphical representation of the 3D model with a visual marker at a location on the 3D model corresponding to the site contacted by the segment of the elongated electrode.

By identifying respective segments of the elongated electrodes that are engaging tissue on the inner surface of the body cavity, systems implementing embodiments of the invention can aid a medical professional to accurately target areas of tissue for treatment. For example, if the elongated electrodes are configured to deliver ablation energy, systems implementing embodiments of the invention can accurately present the visual markers (e.g., ablation tags) that indicate locations in the tissue where the ablation energy is being delivered by the elongated electrodes during an ablation procedure.

### System Description

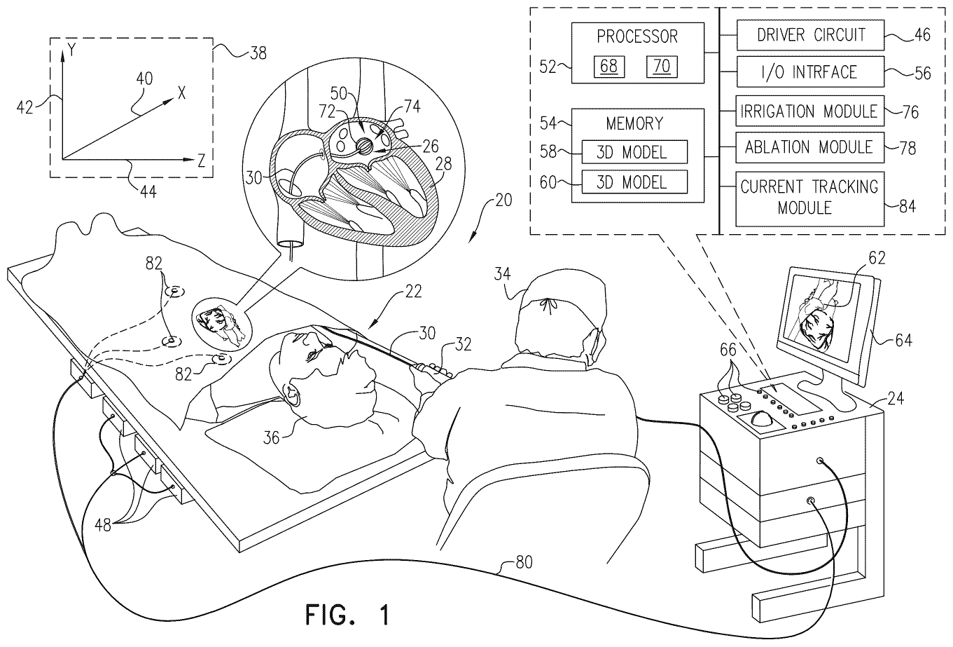

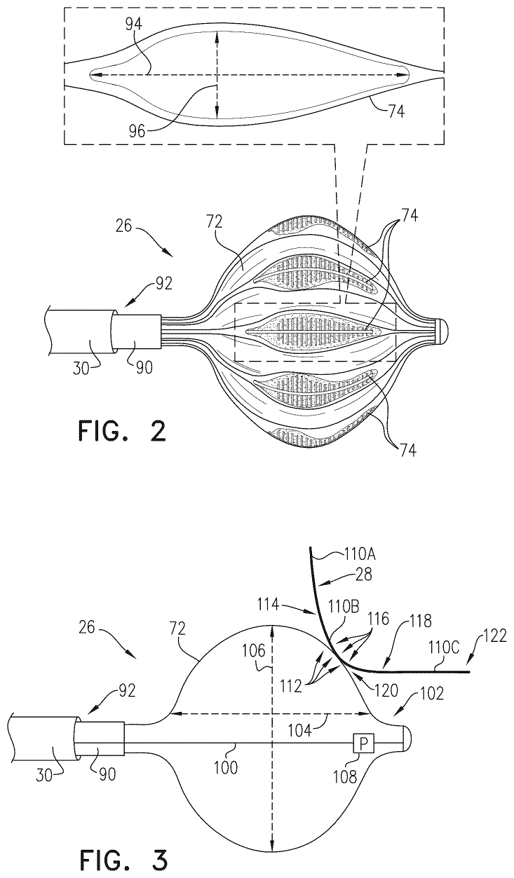

FIG. 1 is a schematic pictorial illustration of a medical system 20 comprising a medical probe 22 (e.g., an intracardiac catheter) and a control console 24, FIG. 2 is a schematic pictorial illustration of a distal end 26 of the medical probe, and FIG. 3 is a schematic cutaway view of the distal end 26, in accordance with an embodiment of the present invention. System 20 may be based, for example, on the CARTO® system, produced by Biosense Webster Inc. (33 Technology Drive, Irvine, CA 92618 USA). In embodiments described hereinbelow, it is assumed that probe 22 is used for diagnostic or therapeutic treatment, such as performing ablation of heart tissue in a heart 28. Alternatively, probe 22 may be used, mutatis mutandis, for other therapeutic and/or diagnostic purposes in the heart or in other body organs.

Probe 22 comprises an insertion tube 30 and a handle 32 coupled to a proximal end of the insertion tube. By manipulating handle 32, a medical professional 34 can insert probe 22 into a body cavity in a patient 36. For example, medical professional 34 can insert probe 22 through the vascular system of patient 36 so that distal end 26 of probe 22 enters a chamber of heart 28 and engages endocardial tissue at a desired location or locations.

In a first embodiment of the configuration shown in FIG. 1, system 20 uses magnetic position sensing to determine position coordinates indicate a location and an orientation of distal end 26 in a coordinate system 38 comprising an X-axis 40, a Y-axis 42 and a Z-axis 44. To implement magnetic based position sensing, control 24 comprises a driver circuit 46 which drives field generators 48 to generate magnetic fields within the body of patient 36. Typically, field generators 48 comprise coils, which are placed below the patient's torso at known positions external to patient 36. These coils generate magnetic fields in a predefined working volume that contains heart 28.

System 20 also comprises a position transducer 50 that is associated with medical probe 22, and a processor 52 in medical console 24. In embodiments described herein, position transducer 50 comprises a component of medical system 20 that generates and conveys position signals indicating a current position (i.e., location and orientation) of distal end 26, and processor 52 is configured to receive and process the conveyed position signals in order to compute, in coordinate system 38, orientation and location coordinates of distal end 26.

In the first embodiment of the configuration shown in FIG. 1, position transducer 50 comprises a magnetic field sensor within distal end 26 of probe 22. In this embodiment, the magnetic field sensor generates electrical signals in response to the magnetic fields from the coils, thereby enabling console 24 to determine the position of distal end 26 within the chamber.

In this embodiment, system 20 measures the position of distal end 26 using magnetic-based sensors. Magnetic position tracking techniques are described, for example, in U.S. Pat. Nos. 5,391,199, 5,443,489, 6,788,967, 5,558,091, 6, 172,499 and 6,177,792. The methods of location sensing described hereinabove are implemented in the above-mentioned CARTO® system and are described in detail in the patents cited above.

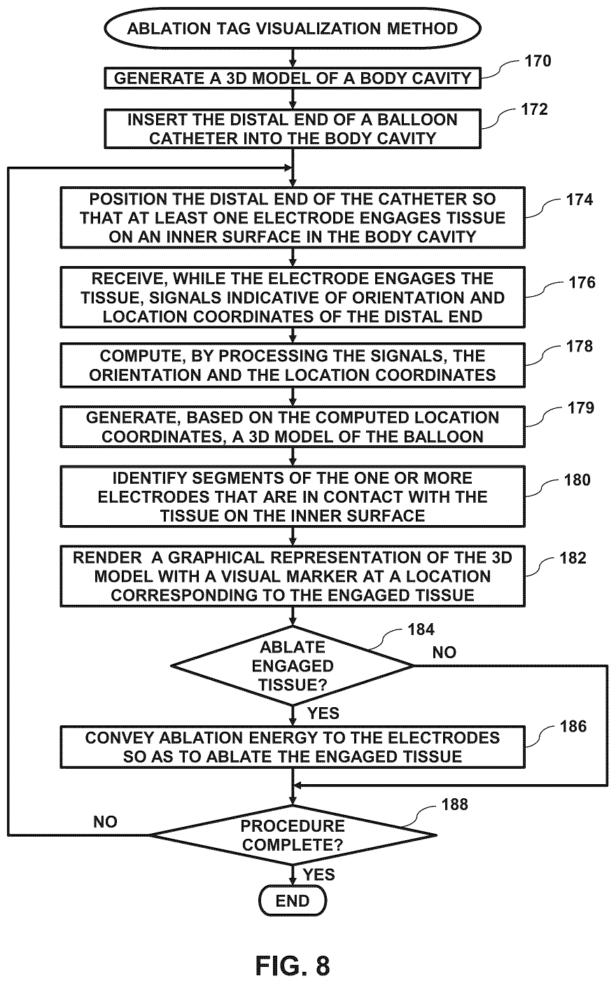

In addition to driver circuit 46 and processor 52, control console 24 also comprises a memory 54 and an input/output (I/O) communications interface 56. In embodiments described herein, memory 54 stores a 3D model 58 of heart 28 that processor 52 can generate based on 3D data such as anatomical mapping data received from a mapping catheter, computed tomography (CT) data, magnetic resonance imaging (MRI) data and ultrasound data. In some embodiments, memory 54 can also store a 3D model 60 of distal end 26, as described in the description referencing FIG. 8 hereinbelow.

In operation, I/O communications interface 56 enables control console 24 to receive signals from position transducer 50. Based on signals received from position transducer 50, processor 52 can process these signals in order to determine the position coordinates of distal end 26, typically comprising both location and orientation coordinates. As described in the description referencing FIG. 8 hereinbelow, processor 52 can update 3D model 58 based on the determined position coordinates.

During the procedure, processor 52 can present, to medical professional 34, a graphical representation 62 of 3D model 58 on a display 64. In some embodiments, medical professional 34 can manipulate graphical representation 62 using one or more input devices 66. In alternative embodiments, display 64 may comprise a touchscreen that can be configured to accept inputs from medical professional 34, in addition to presenting graphical representation 62.

Processor 52 may comprise real-time noise reduction circuitry 68 typically configured as a field programmable gate array (FPGA), followed by an analog-to-digital (A/D) signal conversion integrated circuit 70. The processor can be programmed to perform one or more algorithms disclosed herein, each of the one or more algorithms comprising steps described hereinbelow. The processor uses circuitry 68 and circuit 70 as well as features of modules which are described in more detail below, in order to perform the one or more algorithms.

Memory 54 may comprise any suitable volatile and/or non-volatile memory, such as random access memory, a solid-state drive or a hard disk drive.

Medical probe 22 comprises a balloon 72 having multiple elongated electrodes 74 that can be used to ablate tissue in a body cavity such as heart 28. Balloon 72 and electrodes 74 are described in the description referencing FIG. 2 hereinbelow.

Control console 24 further comprises an irrigation module 76 that controls the inflation of balloon 72 and an ablation module 78 that controls the delivery of ablation energy to elongated electrodes 74.

In a second embodiment of the configuration shown in FIG. 1, medical system 20 may use impedance-based location sensing to determine location coordinates of distal end 26 in coordinate system 38. To implement impedance-based location sensing, control console 24 is connected, by a cable 80, to body surface electrodes, which typically comprise adhesive skin patches 82 that are affixed to patient 36. In the configuration shown in FIG. 1, cable 80 also connects field generators 48 to console 24.

Control console 24 also comprises a current tracking module 84 that, in conjunction with processor 52, determines position coordinates of distal end 26 inside heart 28 based on impedances and/or currents measured between adhesive skin patches 82 and electrodes 74. When using impedance-based location sensing to determine location coordinates of distal end 26 in coordinate system 38, position transducer 50 may comprise a selected electrode 74 operating with adhesive patches 82.

Impedance-based and current-based position tracking techniques are described, for example, in U.S. Pat. Nos. 5,983,126, 6,456,864 and 5,944,022. The methods of position sensing described hereinabove are implemented in the above-mentioned CARTO® system and are described in detail in the patents cited above.

In operation, irrigation module 76 can use irrigation fluid to inflate balloon 72, and can control the inflation of the balloon by controlling a flow rate of the irrigation fluid into the balloon. Balloon 72 is typically formed from bio-compatible material such as polyethylene terephthalate (PET), polyurethane, Nylon, or Pebax. In some embodiments, balloon 72 may comprise multiple small fenestrations (not shown) that allow the irrigation fluid to exit the balloon. These fenestrations are typically 0.025-0.500 millimeters in diameter.

Ablation module 78 is configured to monitor and control ablation parameters such as the level and the duration of ablation power (e.g., radio-frequency energy) conveyed to elongated electrodes 74 via I/O interface 56.

In the configuration shown in FIG. 2, balloon 72 comprises elongated electrodes 74 that are disposed longitudinally on the exterior surface of the balloon, and the balloon is affixed to a tubular shaft 90. Balloon 72 is configured to extend from a distal end of a lumen 92 of insertion tube 30, and the balloon can be deployed through the lumen into a body cavity such as heart 28. For simplicity, connections of elongated electrodes 74 to I/O interface 56 and ablation module 78 are not shown. In some embodiments, the connections are made by wires (not shown) running from the inside of the balloon to the outer surface of the balloon. The electrical connections can be formed with conductive epoxy or welding.

Elongated electrodes 74 can be fabricated with the balloon and typically comprise gold overlaying the exterior wall of balloon 72. In embodiments of the present invention, the elongated electrodes have respective lengths 94 that are at least twice as long as their respective widths 96.

As shown in FIG. 3, medical probe 22 also comprises an extender shaft 100 that is contained within tubular shaft 90, and is coupled to a distal end 102 of balloon 72. In operation, medical professional 34 can control a length 104 of balloon 72 (i.e., once the balloon is deployed from the lumen) by extending or retracting extender shaft 100 and the operator can control a width 106 of the balloon by specifying, to irrigation module 76, the flow rate of the irrigation fluid into the balloon.

In the configuration shown in FIG. 3, a magnetic field sensor 108 that is affixed to extender shaft 100 acts as a position transducer. In operation, processor 52 can process signals received from magnetic field sensor 108 in order to determine, in coordinate system 38, location coordinates of the magnetic field sensor. In some embodiments, as described hereinbelow, processor 52 can determine the current shape of balloon 72 (e.g., length 104 and width 106) based on the determined location coordinates that indicate the length of the balloon and the irrigation fluid flow rate that (i.e., combined with the current length of the balloon) indicates the current width of the balloon.

As shown in FIG. 3, Balloon 72 has a generally spherical shape when inflated. During a medical procedure, medical professional 34 may maneuver distal end 26 so that balloon 72 engages tissue 110 in a body cavity (e.g., heart 28) of patient 36. Balloon 72 can typically retain its generally spherical shape (and not have any distortion in the generally spherical shape) if a force 112 of the irrigation fluid on an inside surface 114 of the balloon is equal to or greater than a force 116 of tissue 110 on an outer surface 118 of the balloon.

In embodiments described hereinbelow, tissue 110 can be differentiated by appending a letter to the identifying numeral, so that the tissue comprises cardiac tissue 110A in heart 28, ostial tissue 110B in a pulmonary vein ostia 120, and intravenous tissue 110C in a pulmonary vein 122.

While the configuration of medical probe 22 presented in FIGS. 1-3 shows the medical probe comprising a balloon catheter having elongated electrodes 74 mounted on balloon 72, using any other type of medical probe 22 comprising any number of elongated electrodes configured to engage tissue in any body cavity in patient 36 is considered to be within the spirit and scope of the present invention.

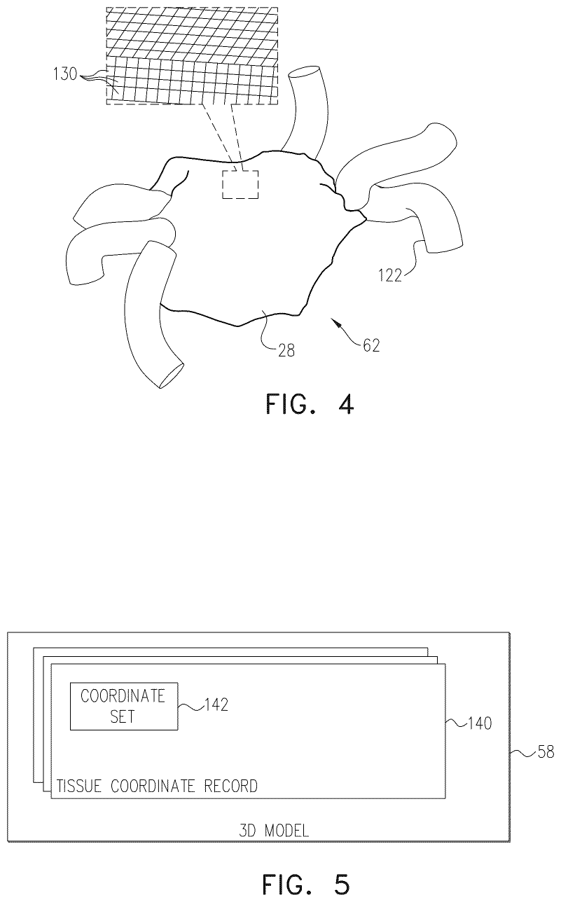

FIG. 4 is a schematic pictorial illustration of voxels 130 that processor 52 can use to generate 3D model 58 of heart 28, in accordance with an embodiment of the present invention. In embodiments described herein, voxels 130 correspond to three-dimensional data points within tissue 110.

To generate model 58, processor 52 receives three-dimensional data for tissue 110, and then segments the received three-dimensional data into a set of voxels 130, wherein each given voxel 130 corresponds to a respective set of 3D location coordinates in coordinate system 38. As described supra, examples of the 3D data include anatomical mapping data received from a mapping catheter, computed tomography (CT) data, magnetic resonance imaging (MRI) data and ultrasound data.

FIG. 5 is a block diagram showing an example of 3D model 58, in accordance with an embodiment of the present invention. In the example shown in FIG. 5, model 58 comprises a plurality of tissue coordinate records 140 having a one-to-one correspondence with voxels 130. Each given record 140 comprises a set of 3D coordinates (i.e., in coordinate system 38) 142 for the corresponding voxel 130.

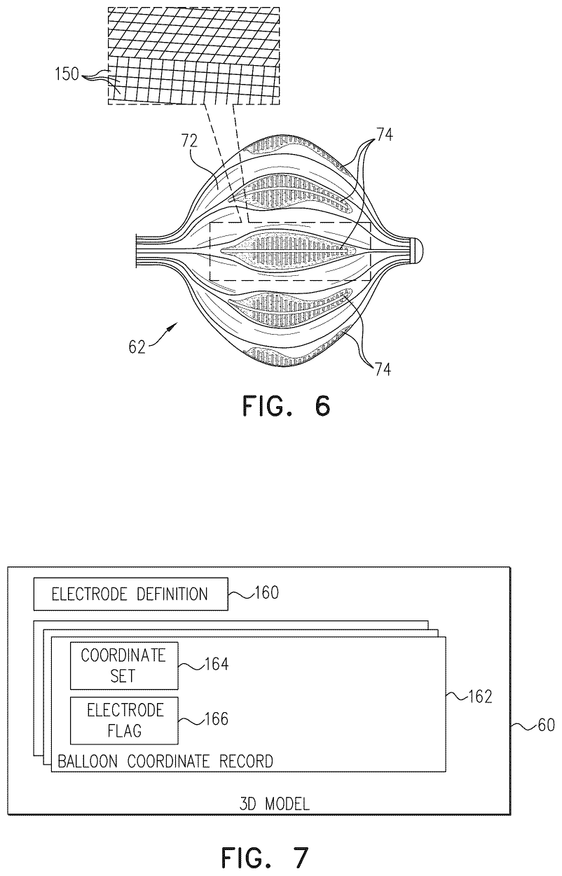

FIG. 6 is a schematic pictorial illustration of voxels 150 that processor 52 can use to generate 3D model 60 of heart balloon 72, in accordance with an embodiment of the present invention. In embodiments described herein, voxels 150 correspond to three-dimensional data points within balloon 72 and elongated electrodes 74.

FIG. 7 is an example of 3D model 60, in accordance with an embodiment of the present invention. In the example shown in FIG. 7, model 60 comprises an electrode definition 160 and a plurality of balloon coordinate records 162 having a one-to-one correspondence with voxels 150. In some embodiments, electrode definition 160 comprises dimensions of electrodes 74.

Each given record 162 comprises a set of 3D coordinates (i.e., in coordinate system 38) 164 for the corresponding voxel 150, and an electrode flag 166. In some embodiments, processor 52 can set electrode flag 166 (e.g., to “True”) in a given record 162 if electrode definition 160 indicates that coordinates 164 in the given record correspond to the coordinates of a given elongated electrode 74.

When balloon 72 is inflated, the location coordinates that processor 52 computes for a given elongated electrode 74 (i.e., based on impedances and/or currents between adhesive skin patches 82 and the given elongated electrode) may comprise the location coordinates of a centroid of the given electrode. The centroid is herein assumed to be on the equator of balloon 72.

As described supra, balloon 72 has a generally spherical shape when inflated. Therefore, processor can use the location coordinates of the centroids of elongated electrodes to “model” balloon 72 so as to identify which voxels 150 correspond to the balloon, as described hereinbelow. In some embodiments, processor 52 can identify which voxels 150 correspond to elongated electrodes 74 based on the location coordinates of the centroids of the electrodes and electrode definition 160.

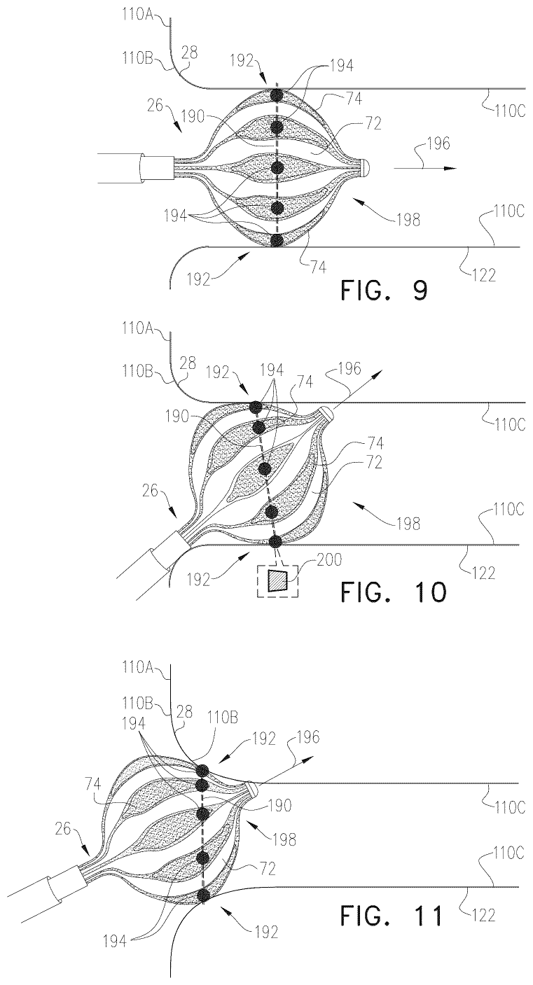

FIG. 8 is a flow diagram that schematically illustrates a method of presenting, on display 64, visual markers that correspond to location coordinates where elongated electrodes 74 engage an inner surface (i.e., tissue) in a body cavity such as heart 28, in accordance with an embodiment of the present invention. In a first model generation step 170, processor 52 generates 3D model 58. For example, processor 52 can generate 3D model 58 of a body cavity such as heart 28 or a pulmonary vein (as shown in FIGS. 9-11 and described hereinbelow) based on mapping points previously acquired by a mapping catheter (not shown). In some embodiments, processor 52 can augment (and increase the accuracy) of 3D model 58 with 3D image data received from a 3D imaging system such as a computed tomography (CT) scanner, a magnetic resonance imaging (MRI) scanner, or an ultrasound scanner.

In an insertion step 172, medical professional 34 inserts distal end 26 of medical probe 22 into the body cavity. Upon inserting distal end into the body cavity, the medical professional can inflate balloon 72 using methods described hereinabove.

In a positioning step 174, the medical professional manipulates handle 32 so that one or more elongated electrodes 74 engage tissue (e.g., tissue 110) on an inner surface of the body cavity. For example, medical professional can position distal end 26 so that at least one elongated electrode 74 engages tissue 110A in heart 28.

In a receive step 176, processor 52 receives, from position transducer 50, signals indicative of an orientation 196 and location coordinates 198 of distal end 26, and in a computation step 178, the processor computes the orientation and the location coordinates (i.e., in coordinate system 38) of the distal end. In a first location computation embodiment, magnetic field sensor 108 conveys, to processor 52, electrical signals in response to the magnetic fields generated by the coils in field generators 48, and upon receiving the electrical signals, the processor can compute a position (i.e., location coordinates and an orientation) of the magnetic field sensor (and thereby the position of distal end 26) within the body cavity.

In a second location computation embodiment, processor 52 determines location coordinates of distal end 26 inside the body cavity based on impedances and/or currents measured between adhesive skin patches 82 and elongated electrodes 74. As described supra, elongated electrodes 74 are disposed longitudinally on the exterior surface of balloon 72. When balloon 72 is inflated, processor 52 can compute (as described supra) the 3D location coordinates for each given elongated electrode 74 as a centroid of the given electrode. In some embodiments, processor 52 can compute an orientation of distal end 26 based on the 3D location coordinates of electrodes 74.

In a second model generation step 179, processor 52 generates model 60 for balloon 72. Since balloon 72 has a generally spherical shape when inflated and elongated electrodes 74 are disposed on the outer surface of the balloon, processor 52 can compute a center of balloon 72 as an average of the location coordinates corresponding to the respective centroids of elongated electrodes 74, and then compute a radius of the balloon based on the computed center and the location coordinates of the electrodes. Using the computed radius and center, processor 52 can then compute sets of 3D location coordinates for balloon 72 in coordinate system 38, segment the computed sets of 3D location coordinates into a set of voxels 150, and store, to the respective balloon coordinate record 162 for each given voxel 150, the computed 3D location coordinates to coordinates 164.

As described supra, distal end comprises balloon 72 and elongated electrodes 74. Based on the location coordinates for the centroids of elongated electrodes 74 and the dimension information stored in electrode definition 160, processor 52 can compute sets of 3D location coordinates for the electrodes, and set the electrode flags in the balloon coordinate records whose coordinates 164 match any of the computed sets of 3D location coordinates for the electrodes. In other words, processor 52 can identify which voxels 150 correspond to the 3D location coordinates of the elongated electrodes.

FIGS. 9-11 are schematic pictorial illustrations showing examples of elongated electrodes engaging tissue on an inner surface of a body cavity such as heart 28, in accordance with an embodiment of the present invention. As shown in FIGS. 9 and 10, medical professional 34 inserts, via a chamber of heart 28, distal end 26 of medical probe 22 into pulmonary vein 122. Upon inserting balloon 72 into pulmonary vein 122 and extending extender shaft 100 into the pulmonary vein, medical professional 34 inflates (i.e., via irrigation module 76) balloon 72 so that respective segments 192 of one or more elongated electrodes 74 engage intravenous tissue 110C at respective engagement areas 194 (also referred to herein as sites), having respective contours 200, as shown in FIG. 10.

While a given elongated electrode 74 engages intravenous tissue 110C (or any other tissue 110 in a body cavity in patient 36), a given segment 192 of the given elongated electrode engages (i.e., is in contact with) the intravenous tissue at a given engagement area 194 on the intravenous tissue, while parts of the given elongated electrode other than the identified segment are not in contact with the intravenous tissue. In an identification step 180, processor 52 identifies respective segments 192 of the elongated electrodes engaging engagement areas 194 on the intravenous tissue.

In one embodiment, processor 52 can detect engagement areas 194 based on models 58 and 60. In this embodiment, processor 52 can use a collision detection algorithm on coordinate sets 142 and 164 to identify which voxels 150 are within a minimum distance threshold to any voxel 130. The identified voxels correspond to the location coordinates for engagement areas 194.

In the examples shown in FIGS. 9 and 10, medical professional 34 inserted distal end 26 into pulmonary vein 122 at different respective orientations 196. Due to the different orientations 196, the segment of a given elongated electrode 74 that engages intravenous tissue 110C in the example shown in FIG. 9 typically differs from the segment of the given elongated electrode that engages the intravenous tissue in the example shown in FIG. 10.

In some embodiments, as described hereinbelow, medical professional can instruct ablation module 78 to deliver ablation energy to elongated electrodes 74, thereby ablating intravenous tissue at engagement areas 194. In these embodiments, a line 190 connecting all engagement areas 194 may also be referred to as ablation line 190.

While the examples in FIGS. 9 and 10 show balloon 72 deployed in pulmonary vein 122, deploying the balloon in other organs of patient 36 and determining the locations of any tissue engaged by elongated electrodes 74 is considered to be within the spirit and scope of the present invention. For example, FIG. 11 shows segments 192 of elongated electrodes 74 engaging ostial tissue 110B at respective engagement areas 194.

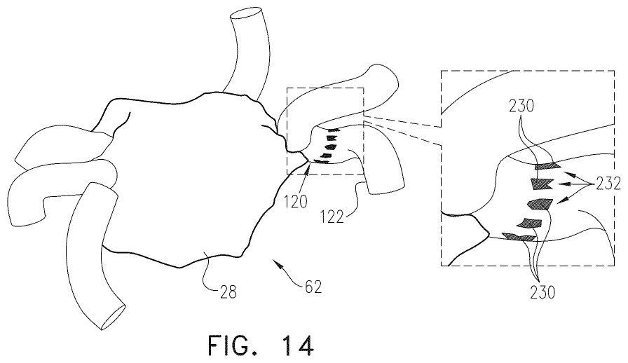

Returning to the flow diagram, in a rendering step 182, processor 52 renders, to display 64, graphical representation 62 of 3D model 58, with visual markers at respective locations in the 3D model corresponding to the engagement areas on intravenous tissue 110C. In some embodiments (as shown in FIGS. 13 and 14 and described hereinbelow), processor 52 can render the visual markers using display contours corresponding to contours 200 of engagement areas 194.

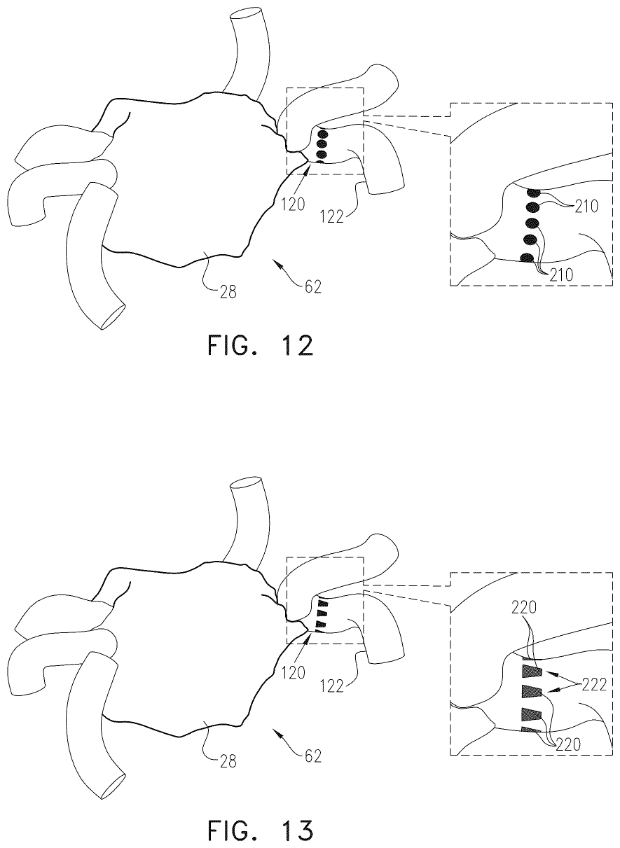

FIG. 12 is a schematic pictorial illustration of graphical representation 62 of 3D model 58 representing heart 28, in accordance with a first embodiment of the present invention. In the first embodiment presented in FIG. 12, processor 52 renders graphical representation 62 with visual markers 210 that have circular display contours and correspond to engagement areas 194 on intravenous tissue 110C. While the example in FIG. 12 shows processor 52 rendering visual markers 210 as circular display contours, presenting the visual markers using other contours is considered to be within the spirit and scope of the present invention.

FIG. 13 is a schematic pictorial illustration of graphical representation 62 of 3D model 58 representing heart 28, in accordance with a second embodiment of the present invention. In the second embodiment presented in FIG. 13, processor 52 renders graphical representation 62 with visual markers 220 that have trapezoidal contours 222 that are similar to contours 200 of their corresponding engagement areas 194.

The examples shown in FIGS. 12 and 13 correspond to orientation 196 of distal end 26 shown in FIG. 9 where the orientation of balloon 72 at the distal end is aligned with pulmonary vein 122. In some embodiments, the orientation of distal end 26 can be considered to be aligned with pulmonary vein 122 when orientation 196 is within 15 degrees of parallel with a section of the pulmonary vein comprising engagement area(s) 194. When balloon 72 is aligned with pulmonary vein 122, visual markers 220 may have similar contours 222, as shown in FIG. 13.

FIG. 14 is a schematic pictorial illustration of graphical representation 62 of 3D model 58 representing heart 28, in accordance with a third embodiment of the present invention. The example shown in FIG. 14 corresponds to orientation 196 of distal end 26 shown in FIG. 10, where the orientation of balloon 72 at the distal end is not aligned with pulmonary vein 122. When balloon 72 is not aligned with pulmonary vein 122, processor 52 can generate visual markers 230 that have differing contours 232 corresponding to contours 200 of their respective engagement areas 194, as shown in the third embodiment.

Returning to the flow diagram, in a first decision step 184, if the engaged intravenous tissue comprises intravenous tissue 110C targeted for ablation, then in an ablation step 186, ablation module 78 conveys ablation energy to elongated electrodes 74, thereby ablating the intravenous tissue engaged by the elongated electrodes. In some embodiments, ablation module 78 can convey ablation energy to elongated electrodes 74 in response to input received from medical professional 34 (e.g., via input devices 66).

In some embodiments, processor 52 can render each given visual marker 210, 220 or 230 in response to ablation module 78 delivering ablation energy to elongated electrodes 74. In these embodiments, the visual markers comprise ablation tags that indicate ablation locations on intravenous tissue 110C.

In a second decision step 188, if the medical procedure is not complete, then the method continues with step 174. If the medical procedure is complete, then the method ends.

Returning to step 184, if the engaged intravenous tissue does not comprise intravenous tissue 110C targeted for ablation, then the method continues with step 188.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

What is claimed is:

1. An apparatus, comprising:
a medical probe configured to be inserted into a body cavity and comprising a distal end having at least one elongated electrode disposed longitudinally along the distal end, the at least one elongated electrode comprising a width and a length, the length being longer than the width;
a position transducer associated with the medical probe;
a memory configured to store a three-dimensional (3D) model of the body cavity;
a display; and
a processor configured:
to receive, from the position transducer, signals indicative of orientation and location coordinates of the distal end within the body cavity,
to identify, based on the 3D model and the signals, a segment along the length of a given elongated electrode that is in contact with tissue at a site on an inner surface of the body cavity, while parts of the given elongated electrode other than the identified segment are not in contact with the inner surface of the body cavity, and
to render to the display a graphical representation of the 3D model with a visual marker at a location on the 3D model corresponding to the site contacted by the segment of the given elongated electrode.

2. The apparatus according to claim 1, the length of the at least one elongated electrode being at least twice as long as the width of the at least one elongated electrode.

3. The apparatus according to claim 1, and comprising an inflatable balloon that extends from a lumen in the distal end of the medical probe.

4. The apparatus according to claim 3, the at least one elongated electrode being disposed longitudinally on a surface of the balloon, the length of the at least one elongated electrode being at least twice as long as the width of the at least one elongated electrode.

5. The apparatus according to claim 1, and further comprising an ablation module configured to deliver ablation energy to the at least one elongated electrode, thereby ablating the tissue that is in contact with the at least one electrode.

6. The apparatus according to claim 1, the processor being further configured:
to receive, prior to receiving the signals, 3D model data for the body cavity, the 3D model data comprising a first set of voxels, and
to generate, using the first set of voxels, the 3D model.

7. The apparatus according to claim 6, the location coordinates of the distal end comprising location coordinates of a centroid of the given elongated electrode.

8. The apparatus according to claim 7, the processor being further configured:
to generate, using the centroid of the given elongated electrode, sets of 3D location coordinates of the balloon;
to segment the computed sets of 3D location coordinates into a second set of voxels; and
identify, using the centroid of the given elongated electrode, which voxels of the second set of voxels correspond to 3D location coordinates of the elongated electrodes.

9. The apparatus according to claim 8, the processor being configured to identify, based on the 3D model and the signals, the segment along the length of the given elongated electrode that is in contact with tissue by identifying voxels of the second set of voxels that are within a minimum distance threshold to any voxel of the first set of voxels.

10. A method, comprising:
generating a three-dimensional (3D) model of a body cavity;
receiving, from a position transducer associated with a medical probe configured to be inserted into the body cavity and comprising a distal end having at least one elongated electrode disposed longitudinally along the distal end, signals indicative of orientation and location coordinates of the distal end within the body cavity, the at least one elongated electrode comprising a width and a length, the length being longer than the width;
identifying, based on the 3D model and the signals, a segment along a length of a given elongated electrode that is in contact with tissue at a site on an inner surface of the body cavity, while parts of the given elongated electrode other than the identified segment are not in contact with the inner surface of the body cavity; and
rendering to the display a graphical representation of the 3D model with a visual marker at a location on the 3D model corresponding to the site contacted by the segment of the given elongated electrode.

11. The method according to claim 10, the length of the at least one elongated electrode being at least twice as long as the width of the at least one elongated electrode.

12. The method according to claim 10, the medical probe comprising an inflatable balloon that extends from a lumen in the distal end of the medical probe.

13. The method according to claim 12, the at least one elongated electrode being disposed longitudinally on a surface of the balloon and the length of the at least one elongated electrode being at least twice as long as the width of the at least one elongated electrode.

14. The method according to claim 10, and further comprising delivering, by an ablation module, ablation energy to the at least one elongated electrodes, thereby ablating the tissue that is in contact with the at least one electrode.

15. The method according to claim 14, the visual marker corresponding to the site ablated by the segment of the given elongated electrode.

16. The method according to claim 10, and comprising:
receiving, prior to receiving the signals, 3D model data for the body cavity, the 3D model data comprising a first set of voxels, and
generating, using the first set of voxels, the 3D model.

17. The method according to claim 16, the location coordinates of the distal end comprising location coordinates of a centroid of the given elongated electrode.

18. The method according to claim 17, and comprising:
generating, using the centroid of the given elongated electrode, sets of 3D location coordinates of the balloon;
segmenting the computed sets of 3D location coordinates into a second set of voxels; and
identifying, using the centroid of the given elongated electrode, which voxels of the second set of voxels correspond to 3D location coordinates of the elongated electrodes.

19. The method according to claim 18, and comprising:
identifying, based on the 3D model and the signals, the segment along the length of the given elongated electrode that is in contact with tissue by identifying voxels of the second set of voxels that are within a minimum distance threshold to any voxel of the first set of voxels.

20. A computer software product, operated in conjunction with a medical probe configured to be inserted into a body cavity and comprising a distal end having at least one elongated electrode disposed longitudinally along the distal end, the at least one elongated electrode comprising a width and a length, the length being shorter than the width, the product comprising a non-transitory computer-readable medium, in which program instructions are stored, which instructions, when read by a computer, cause the computer:
to generate a three-dimensional (3D) model of a body cavity;
to receive, from a position transducer associated with the medical probe, signals indicative of orientation and location coordinates of the distal end within the body cavity;
to identify, based on the 3D model and the signals, a segment along a length of a given elongated electrode that is in contact with tissue at a site on an inner surface of the body cavity, while parts of the given elongated electrode other than the identified segment are not in contact with the inner surface of the body cavity; and
to render to the display a graphical representation of the 3D model with a visual marker at a location on the 3D model corresponding to the site contacted by the segment of the given elongated electrode.
